(19) United States
(12) Patent Application Publication
(10) Pub. No.: US 2023/0221221 A1
(43) Pub. Date: Jul. 13, 2023

(54) TISSUE CORING AND ANALYSIS SYSTEM

(71) Applicant: TriMetis Life Sciences, LLC, Memphis, TN (US)

(72) Inventors: Leif E. Honda, Rye, NH (US); Jon C. Wetzel, Germantown, TN (US); Phil A. Cestaro, Memphis, TN (US)

(73) Assignee: TriMetis Life Sciences, LLC, Memphis, TN (US)

(21) Appl. No.: 18/153,253

(22) Filed: Jan. 11, 2023

Related U.S. Application Data
(60) Provisional application No. 63/266,673, filed on Jan. 11, 2022.

(51) Int. Cl. G01N 1/31; C12Q 1/6806

(52) U.S. Cl. CPC G01N 1/31; C12Q 1/6806; G01N 2001/2873

(57) ABSTRACT

A high-throughput method of analyzing multiple tissue samples for nucleic acid material is presented. The method includes providing FFPE blocks containing tissue samples, a heatmap of locations of interest for each sample-containing FFPE block, and a coring device with a hollow punch tip. The sample-containing FFPE block is punched with the hollow punch tip at a location determined by the heatmap in order to obtain a core sample which is subsequently ejected out of the hollow punch tip to a designated receptacle. The hollow punch tip is sterilized after ejecting the core sample. These steps are repeated as needed to obtain desired number and typed core samples from the sample-containing FFPE blocks. The individual core samples are then treated in the individual designated receptacles with an appropriate reagent to extract nucleic acid material which is subsequently isolated and analyzed.

### RELATED APPLICATIONS

The present application claims benefit under 35 U.S.C. 119(e) from earlier filed U.S. Provisional Application No. 63/266,673, filed Jan. 11, 2022, which is incorporated herein by reference in its entirety for all purposes.

### BACKGROUND

Delays in diagnosis, decision and treatment of diseases and genetic conditions can arise from the manual preparation, multiple material transfers, and human visual microscopic observation needed in current processes to extract and analyze samples for pathological, immunohistochemical, and genomic information.

The present disclosure relates to high-throughput method for analyzing tissue samples of nucleic acid material disposed on a Formalin Fixed Paraffin Embedded (“FFPE”) block is disclosed, and will provide analyses without undue delays.

### SUMMARY

This Summary is provided to introduce a selection of concepts in a simplified form that is further described below in the Detailed Description. This Summary is not intended to identify key features or essential features of the claimed subject matter, nor is it intended to be used to limit the scope of the claimed subject matter.

A high-throughput method for analyzing tissue samples of nucleic acid material disposed on a Formalin Fixed Paraffin Embedded (FFPE) block is disclosed. The method involves generating, with a computer, a heatmap of locations of interest for the FFPE block and then obtaining core samples from each location of interest in the heatmap by sterilizing a hollow punch tip, punching a respective location of interest with the hollow punch tip to pick up a respective core sample, and depositing the respective core sample to a respective receptacle.

The presently disclosed method further includes treating the core samples deposited in the receptacles with an appropriate reagent to extract the nucleic acid material, then isolating the extracted nucleic acid material, and analyzing the isolated nucleic acid material.

The presently disclosed method utilizes the TriMetis Computer-assisted Pathology (“TCAP”) system for identifying various characteristics in the Formalin Fixed Paraffin Embedded (“FFPE”) tissue blocks. The presently disclosed method can also analyze tissue samples presented in forms or fixates other than FFPE blocks, such as, without limitation, fresh tissue, preserved tissue using fixatives other than formalin, such as, Weigners, Greenfix, UPM, CyMol, Bouin and Hollande. In some embodiments, the FFPE block may be a slide, a substrate or other sample holders.

Bar-coded digital Images are utilized to identify pathological and morphological features of the tumor including the location of tumor, presence and count of tumor nuclei, and the concentration of tumor nuclei and other features.

From these images and Ai output, the TCAP system generates a heatmap that identifies the region of interest. The heatmap is set by the end-user to the desired selection criteria. The result of the criteria identifies the locations of interest, or the target core.

The presently disclosed system can utilize images of both stained and unstained tissue slides or blocks, identify the high density tumor locations, and then collect (extract) the identified tumor materials for downstream DNA or other testing and analysis.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1 illustrates an exemplary method according to various embodiments, and

FIG. 2A shows a scanned H&E image; FIG. 2B is heatmap of locations of interest; FIG. 2C shows high-throughput FFPE blocks; FIG. 2D shows an automated tissue puncher; FIG. 2E shows a multi-tissue, multi-arm robotic system; FIG. 2F illustrates the process of punching a core sample; FIG. 2G illustrates an array of test tubes; FIG. 2H illustrates a nucleic acid extractor, and FIG. 2I illustrates a genome sequencer which all are representative of the presently disclosed system according to one embodiment of the present disclosure.

### DETAILED DESCRIPTION

According to several embodiments of the presently disclosed method for analyzing tissue samples of nucleic acid material disposed on a Formalin Fixed Paraffin Embedded (FFPE) block, the method involves generating, with a computer, a heatmap of locations of interest for the FFPE block and then obtaining core samples from each location of interest in the heatmap. The samples are obtained by sterilizing a hollow punch tip, punching a respective location of interest with the hollow punch tip to pick up a respective core sample, and depositing the respective core sample to a respective receptacle. Further steps include treating the core samples deposited in the receptacles with an appropriate reagent to extract the nucleic acid material, then isolating the extracted nucleic acid material, and analyzing the isolated nucleic acid material.

The tissue samples analyzed by the method can include human or animal tissue, and can be studied for the presence of carcinogenic tissue, a tumor, and a necrotic tissue, to name a few examples.

One way the depositing step can be performed is by ejecting the respective core sample with a plunger, which can be a mechanical plunger or a pneumatic impulse.

The presently disclosed method can be conducted with unstained FFPE blocks, or in some embodiments the FFPE block can be stained with a stain selected from one or more of Hematoxylin and Eosin (H&E), Immunohistochemistry (IHC), Fluorescence In-situ Hybridization (FISH), Chromogenic In-situ Hybridization (CISH), Spectral Imaging, Confocal Microscopy and other simulated staining techniques.

As set forth in more detail below, the presently disclosed method can utilize information to generate a heatmap with the heatmap being generated by the computer using a machine learning system trained to identify a carcinogenic tissue, a tumor, or a necrotic tissue on the FFPE block.

According to some embodiments of the present method, tracing of the FFPE blocks, the heatmap, and the receptacles can be carried out by unique identifiers, such as barcodes, QR codes, or other similar machine readable identifiers. The heatmap can include an X and Y coordinate for each of the locations of interest on the FFPE block. In some embodiments, the heatmap can analyze for, and provide, X, Y, and Z coordinates, or three dimensional images, for the locations of interest on the tissue sample. In other embodiments, the Z coordinate may be set to zero.

The presently disclosed method can also analyze tissue samples presented in other forms or fixates than FFPE blocks, such as, without limitation, fresh tissue, or preserved tissue using fixatives other than formalin, such as, Weigners fixative, Greenfix, UPM, CyMol, Bouin and Hollande. In some instances, the tissue sample to be analyzed can be embedded in a non-paraffin based substrate, or cryogenically preserved.

According to other embodiments of the present disclosure, a system to analyze tissue samples of nucleic acid material disposed on a Formalin Fixed Paraffin Embedded (FFPE) block including a computer to generate a heatmap of locations of interest for the FFPE block, an automated punching system to obtain core samples from each location of interest in the heatmap by sterilizing a hollow punch tip, punching a respective location of interest with the hollow punch tip to pick up a respective core sample, and depositing the respective core sample to a respective receptacle is taught.

The presently disclosed system further includes a nucleic acid extractor and a nucleic acid analyzer. The nucleic acid extractor can treat the core samples deposited in the receptacles with an appropriate reagent to extract the nucleic acid material, and isolate the extracted nucleic acid material. The nucleic acid analyzer, such as a genome sequencer, can be used for analyzing the isolated nucleic acid material. In some embodiments of the presently disclosed system, the receptacles receiving the core samples may include solutions of the appropriate reagents and/or solvents to initiate the nucleic acid extraction or, in some instances, preserve the core sample for subsequent analysis and characterization.

The tissue samples analyzed by this system can include, for example, human or animal tissue, and can be studied for the presence of carcinogenic tissue, a tumor, and a necrotic tissue.

One way the depositing step can be performed is by ejecting the respective core sample with a plunger, which can be a mechanical plunger or a pneumatic impulse.

The presently disclosed system can utilize unstained FFPE blocks, or in some embodiments the FFPE block can be stained with a stain selected from one or more of Hematoxylin and Eosin (H&E), Immunohistochemistry (IHC), Fluorescence In-situ Hybridization (FISH), Chromogenic In-situ Hybridization (CISH), Spectral Imaging, Confocal Microscopy and other simulated staining techniques.

The presently disclosed system and method can utilize a heatmap the generation of which is performed by a computer using a machine learning system trained to identify a carcinogenic tissue, a tumor, or a necrotic tissue on the FFPE block. The machine learning system is further described in pending patent application Ser. No. 17/449,727. The heatmap can comprise X, Y, and Z coordinates for each of the locations of interest on the FFPE block.

Unique identifiers, such as bar codes or QR codes, can be used to trace and identify the FFPE blocks, the heatmap, and the receptacles. In some embodiments of the presently disclosed system, the system also records any or all of the punch locations of each core sample, the tissue obtained, treatment of the core samples, and results obtained.

Corresponding bar-coded FFPE blocks can be loaded into the instrument. The FFPE blocks are moved to slots for punching. The heatmap is checked for orientation and associated with coordinates on a xyz plane. These coordinates are sent to a robotic arm that selects a sterile hollow coring tip. The hollow punch tip is brought to locations of interest as directed by the heatmap. Then the robotic arm depresses and cores with the hollow punch tip the FFPE block. The core is removed and carried to the corresponding tray with bar-coded tubes for downstream processing.

A used hollow punch tip can be returned to discard into a waste bin. The robot tip holder can also be dipped in a disinfectant bath to clean any remanent DNA. This procedure removes any possibility for cross-contamination. The robot head then moves to another tray to pick up a new hollow punch tip for the next core.

Multiple instrument configurations allow for single to high-volume coring of blocks utilizing single or multiple robots, continuous loading or block autoloaders.

Post coring trays can be located on a conveyor belt. According to some embodiments of the present disclosure, the trays can hold arrays of 24, 48, 96, etc. of test tubes or other suitable sample holders. The arrays are arranged such that the core sample held in the array can be traced back to the FFPE block, heatmap, and coring position. Once a tray is full, it can be moved to a dock where the tray remains until a technician removes it for testing, or if configured appropriately the tray will move to the next processing step, for example, dissociation for genomic testing.

One purpose of the presently disclosed system is to streamline and expedite the process of accessing the highest concentration of tissue for downstream molecular, genomic and DNA testing, which is currently a manual process.

The presently disclosed system removes several time-consuming and wasteful manual sample preparation steps including cutting multiple unstained slides, manual estimation by a highly trained pathologist to circle with a marker the highest concentration of DNA on an H&E (“Haemotoxylin and Eosin”) stained slide, technician micro dissecting or scraping slide for material of interest, and discarding unstained slide waste.

Some additional advantages of the presently disclosed system are significant savings are realized by reducing costs for materials such as preparation reagents, glass slides, razor blades, etc., and requiring fewer high-value low-supply histotechnologists and pathologists for mundane lower-level jobs.

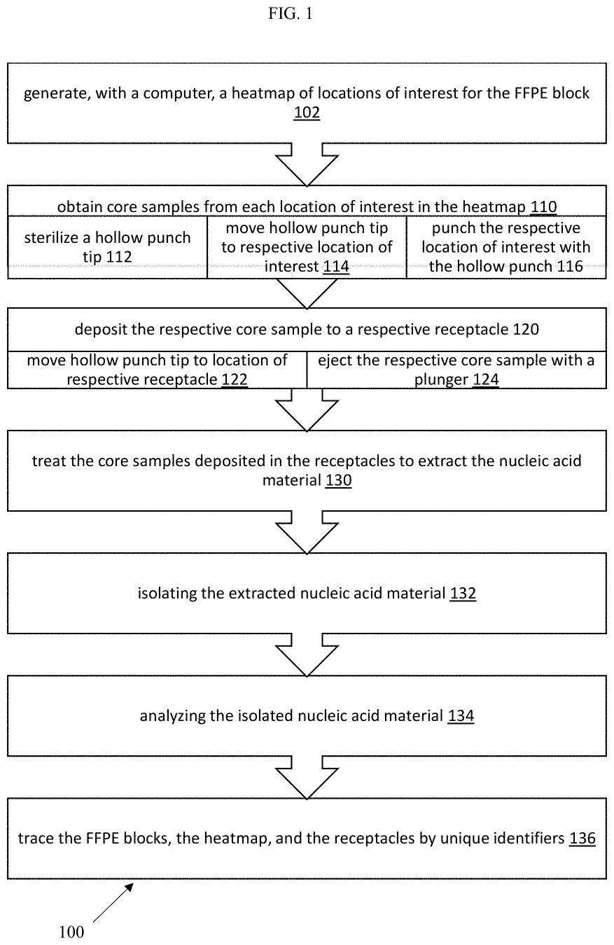

FIG. 1 illustrates an exemplary method for a high-throughput method for analyzing tissue samples of nucleic acid material disposed on a Formalin Fixed Paraffin Embedded (FFPE) block according to various embodiments.

A method 100 for analyzing tissue samples of nucleic acid material disposed on a Formalin Fixed Paraffin Embedded (FFPE) block may include operation 102 to generate, with a computer, a heatmap of locations of interest for the FFPE block. This operation can be followed by operation 110 to obtain core samples from each location of interest in the heatmap. This operation 110 can include three sequential methods 112, 114 and 116, to sterilize a hollow punch tip, move the hollow punch tip to respective location of interest, and then punch the respective location of interest with the hollow punch.

Operation 120 of depositing the respective core sample to a respective receptacle follows obtaining the core sample, and may include operation 122 to move the hollow punch tip to location of respective receptacle, and then operation 124 of ejecting the respective core sample with a plunger. In some embodiments, the plunger can be replaced with a pneumatic impulse driver.

The treating of the core samples deposited in the receptacles to extract the nucleic acid material occurs in operation 130, and is followed by the isolating of the extracted nucleic acid material and the analyzing of the isolated nucleic acid material in operations 132 and 134, respectively. Throughout method 100, operation 136, tracing of the FFPE blocks, the heatmap, and the receptacles by unique identifiers, such as barcodes or QR codes, is enabled.

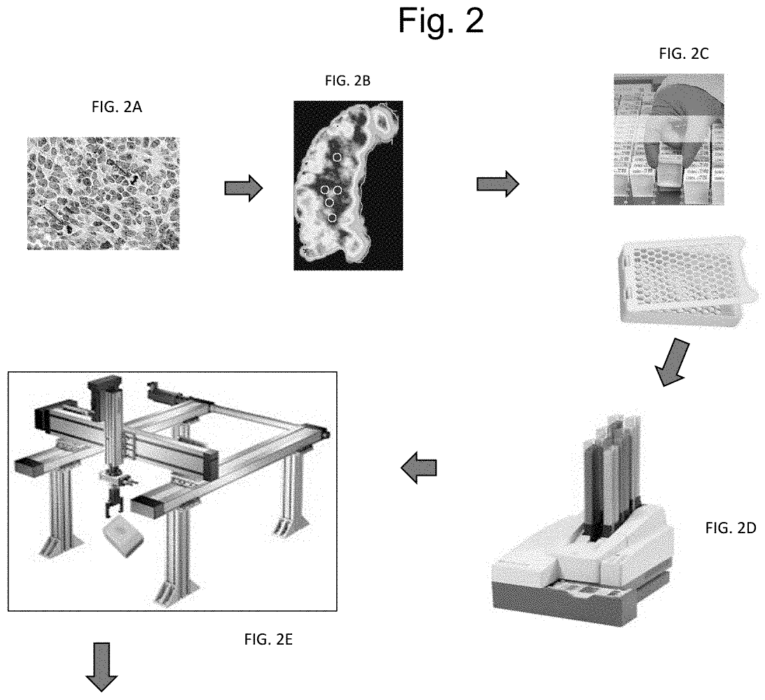

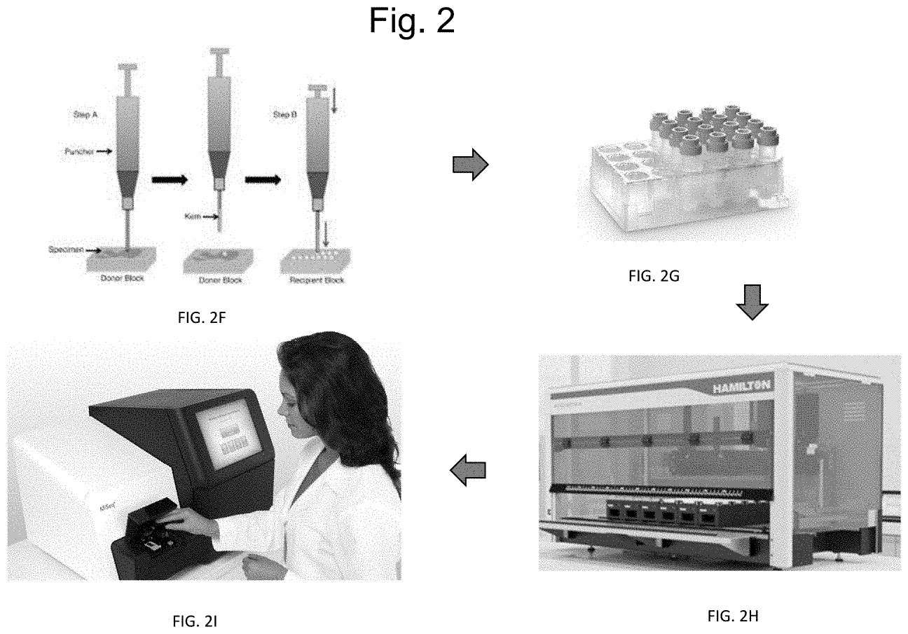

FIG. 2A shows a scanned H&E image that can be analyzed to generate, with a computer, a heatmap of locations of interest for the FFPE block as shown in FIG. 2B. High-throughput FFPE blocks are shown in FIG. 2C. The blocks are then punched in an automated tissue puncher shown in FIG. 2D. A multi-tissue, multi-arm robotic system as shown in FIG. 2E can be utilized in some embodiments of the presently disclosed system to further increase processing rates. FIG. 2F shows the process of punching a core sample from a donor block and then depositing same into the recipient block, in some embodiments of the presently disclosed system core samples are not inserted into recipient blocks but rather are deposited into designated receptacles for subsequent nucleic acid analysis. FIG. 2G illustrates an array of test tubes as possible receptacles for the core samples. An example of a nucleic acid extractor is presented in FIG. 2H. The nucleic acid extracted by the extractor of FIG. 2H can be subsequently genome sequenced as shown in FIG. 21. Not all of the illustrated aspects of FIGS. 2A-2I may be present in all embodiments of the presently disclosed system.

The presently disclosed system can increase throughput by adding processing 24×7×365, each instrument can increase production superior to current human manual processes, multiple instruments can function simultaneously for higher volume, and higher accuracy of quantifiable DNA identification equals higher success rate. The system can be superior to the current human gated manual processes.

The presently disclosed system can be used in conjunction with the TCAP system further described in pending patent application Ser. No. 17/449,727 filed Oct. 1, 2021, entitled “Identifying Morphologic, Histopathologic, and Pathologic Features with a Neural Network,” and published on Apr. 7, 2022, as U.S. Patent Application Publication No. 2022/0108442 A1, which is incorporated herein by reference in its entirety.

The output of the TCAP system allows for the identification of the area of the tumor tissue that has the highest concentration of nuclei, which in turn is an indication of the DNA available for subsequent immunohistochemistry and genomic sequencing.

Any tissue test regime that would benefit from the increased accuracy and quantification that results from the TCAP in combination with the presently disclosed system can utilize these teachings. The presently disclosed system can be applied to veterinary applications in addition to human. The identification, selection, and picking of tissue elements can provide faster processing for downstream processes such as tissue dissociation or cellular isolation.

Known and commercially available TMA systems still utilize a manual process for identifying and punching FFPE blocks for Tissue Microarrays. The presently disclosed system can allow for manual punching of the FFPE but differs on many other levels as the known systems are limited to individual punches. The machine learning identification of locations of interest on the tissue sample provided by the TCAP process leads to higher throughput. The known systems are also not designed for high-throughput analyses, nor for placement of samples into tissue dissociation cartridges. Furthermore, known systems do not utilize Ai or machine learning to handle the identification of the region of interest with the highest tumor nuclei density, nor are known systems capable or designed to handle multiple tissue assays in a production workflow.

The present disclosure will be of interest to molecular genomic profiling entities, pathology equipment manufacturers and pathology diagnostic entities, and addresses the slow manual process of extracting tumor tissue (or any other tissues of concern) and identification of areas of interest.

The presently disclosed system can be utilized in the production for throughput of a molecular testing or diagnostic laboratory. Grunkin et al. provide background information in their U.S. Pat. Nos. 9,697,582 and 10,209,165. The presently disclosed system can be interfaced with various intermediate networks, wireless or wired communications equipment, and the internet and the like to allow for interaction and/or control by parties located physical away from the system.

The present teachings may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as SMALLTALK, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

All publications, articles, papers, patents, patent publications, and other references cited herein are hereby incorporated by reference herein in their entireties for all purposes.

Although the foregoing description is directed to the preferred embodiments of the present teachings, it is noted that other variations and modifications will be apparent to those skilled in the art, and which may be made without departing from the spirit or scope of the present teachings.

The foregoing detailed description of the various embodiments of the present teachings has been provided for the purposes of illustration and description. It is not intended to be exhaustive or to limit the present teachings to the precise embodiments disclosed. Many modifications and variations will be apparent to practitioners skilled in this art. The embodiments were chosen and described in order to best explain the principles of the present teachings and their practical application, thereby enabling others skilled in the art to understand the present teachings for various embodiments and with various modifications as are suited to the particular use contemplated. It is intended that the scope of the present teachings be defined by the following claims and their equivalents.

What we claim is:

1. A high-throughput method for analyzing tissue samples of nucleic acid material disposed on a Formalin Fixed Paraffin Embedded (FFPE) block comprising:
generating, with a computer, a heatmap of locations of interest for the FFPE block; and
obtaining core samples from each location of interest in the heatmap by sterilizing a hollow punch tip,
punching a respective location of interest with the hollow punch tip to pick up a respective core sample, and
depositing the respective core sample to a respective receptacle.

2. The method according to claim 1, further comprising:
treating the core samples deposited in the receptacles with an appropriate reagent to extract the nucleic acid material;
isolating the extracted nucleic acid material; and
analyzing the isolated nucleic acid material.

3. The method according to claim 1, wherein the tissue samples comprise human or animal tissue.

4. The method according to claim 1, wherein the tissue samples comprise a carcinogenic tissue, a tumor, and a necrotic tissue.

5. The method according to claim 1, wherein the depositing is performed by ejecting the respective core sample with a plunger.

6. The method according to claim 1, wherein the FFPE block is unstained.

7. The method according to claim 1, wherein FFPE block is with a stain selected from one or more of Hematoxylin and Eosin (H&E), Immunohistochemistry (IHC), Fluorescence In-situ Hybridization (FISH), Chromogenic In-situ Hybridization (CISH), Spectral Imaging, Confocal Microscopy and a simulated stain.

8. The method according to claim 1, wherein the generating performed by the computer uses a machine learning system trained to identify a carcinogenic tissue, a tumor, or a necrotic tissue on the FFPE block.

9. The method according to claim 1, further comprising tracing the FFPE blocks, the heatmap, and the receptacles by unique identifiers.

10. The method of claim 1, wherein the heatmap comprises an X and Y coordinate for each of the locations of interest on the FFPE block.

11. A system to analyze tissue samples of nucleic acid material disposed on a Formalin Fixed Paraffin Embedded (FFPE) block comprising:
a computer to generate a heatmap of locations of interest for the FFPE block; and
an automated punching system to obtain core samples from each location of interest in the heatmap by
sterilizing a hollow punch tip,
punching a respective location of interest with the hollow punch tip to pick up a respective core sample, and
depositing the respective core sample to a respective receptacle.

12. The system according to claim 11, further comprising:
a nucleic acid extractor; and
a nucleic acid analyzer.

13. The system according to claim 11, wherein the tissue samples comprise human or animal tissue.

14. The system according to claim 11, wherein the tissue samples comprise a carcinogenic tissue, a tumor, and a necrotic tissue.

15. The system according to claim 11, wherein the depositing is performed by ejecting the respective core sample with a plunger.

16. The system according to claim 11, wherein the FFPE block is unstained.

17. The system according to claim 11, wherein FFPE block is with a stain selected from one or more of Hematoxylin and Eosin (H&E), Immunohistochemistry (IHC), Fluorescence In-situ Hybridization (FISH), Chromogenic In-situ Hybridization (CISH), Spectral Imaging, Confocal Microscopy and other simulated staining techniques.

18. The system according to claim 11, wherein the generating performed by the computer uses a machine learning system trained to identify a carcinogenic tissue, a tumor, or a necrotic tissue on the FFPE block.

19. The system according to claim 11, further comprising unique identifiers to trace and identify the FFPE blocks, the heatmap, and the receptacles.

20. The system according to claim 11, wherein the heatmap comprises an X and Y coordinate for each of the locations of interest on the FFPE block.
